(19) United States
(12) Patent Application Publication
(10) Pub. No.: US 2009/0318757 A1
(43) Pub. Date: Dec. 24, 2009

(54) Flexible visually directed medical intubation instrument and method

(75) Inventor: Errol O. Singh, Upper Arlington, OH (US)

Correspondence Address: GALLAGHER & DAWSEY CO., L.P.A., P.O. BOX 785, COLUMBUS, OH, 43216, US

(73) Assignee: PercuVision, LLC

(21) Appl. No.: 12/214,944

(22) Filed: Jun. 23, 2008

(51) Int. Cl. A61B 1/04

(52) U.S. Cl. 600/109

(57) ABSTRACT

A flexible medical intubation instrument provided for placement into an animal or human patient comprises a catheter with at least a pair of longitudinally extending lumens or channels including a sensor and/or actuator channel and a working channel. In the sensor/actuator channel is provided a fixed or slideably removable sensor cable having a sensor for sensing a characteristic or condition including any of the following: a visual sensor for optical viewing, a chemical sensor, a pH sensor, a pressure sensor, an infection sensor, an audio sensor, or a temperature sensor. The sensors are coupled by the sensor/actuator cable through light transmission, electric current, or radio transmission to a viewing instrument or other output device such as a meter or video screen for displaying the condition that is sensed within the body of the patient while the flexibility of the composite structure comprising the catheter and cable enable the entire instrument to flex laterally as it moves through curved passages or around obstructions during insertion or removal. While making observations through the sensor channel, the working channel simultaneously functions as a drain or an irrigation duct, a feeding tube, or to provide a passage for the insertion of one or a succession of surgical devices such that the catheter serves as a protective artificial tract or liner as surgical devices are inserted and removed through it in succession so as to minimize tissue trauma, infection, and pain experienced by the patient. The instrument can be used in urology, as well as a visually directed nasogastric tube, as a visually directed external gastrostomy tube, or as a visually directed internal gastric tube or percutaneous endoscopic gastrostomy tube and in other applications.

### FIELD OF THE INVENTION

This invention relates to medical instrumentation and more particularly to a method and apparatus for facilitating intubation of an animal or human patient.

### BACKGROUND OF THE INVENTION

In many medical procedures it is often necessary to place an instrument into the body of the patient for drainage, for viewing a part of the body, or for performing a surgical operation such as the endoscopic removal of a tumor, to take a biopsy, or for feeding the patient. The invention has general application in medicine including the field of urology as well as in the field of gastroenterology and in other medical and surgical specialties. The placement of a catheter in the urethra for the purpose of draining urine or for diagnostic purposes, for example, is one of the most common urological procedures for draining urine or fluid to determine the amount of urine present, to diagnose problems, or to maintain anatomic continuity. This procedure is commonly performed by inserting the catheter manually while noting any resistance to forward movement as shown by a failure of the catheter to slide smoothly into the urethra. While most placements proceed without problems, typically more than forty percent of male urinary catheter placements are difficult because of the problematic normal anatomy of the male lower urinary tract such as the external sphincter, the S-curve of the bulbous urethra and angulated prostatic urethra and/or pathologic conditions, such as urethral stricture disease, stones, trauma, tumors, enlarged prostate, iatrogenic false passages, and/or congenital disorders causing a substantial burden on the delivery of effective care through the healthcare system. The most common problem is tetany, a spasm of the external urinary sphincter or stricture of the urethra. Stones, and even clots descending from the bladder, also constitute urethral obstructions. In addition, urethral lumen calibers vary considerably, and particularly with urethritis, BPH, urethritis stricture disease and prostate disorders in males. These costs to the healthcare system, hospitals, clinics and doctors' offices are substantial. In addition, the delay in servicing urological catheter patients in a timely manner constitutes poor medical efficiency, delivery, and control. When difficulty is encountered, the resulting frustration among healthcare professionals, especially nurses, physician extenders and physician assistants, creates a very real feeling of ineffectiveness on the part of these healthcare workers, to say nothing of the dissatisfaction on the part of the patients caused by the delay and added discomfort. Difficult catheterizations can also be a source of urinary tract infection. While the dollar cost to the healthcare system is not the only concern, such elements as added labor and material costs, time delays for patient rectification, excess space and equipment required, catheter kit value, nurse technician and physician costs constitute an expense to the healthcare system of surprising proportions. The best available current data indicates about 55,000 urinary catheter placements are made in the United States per day. Of these, conservatively about 40% are difficult which means that they require multiple advances and pull-backs of the urinary catheter to negotiate the urethra, multiple catheters on the same patient, several staff workers attending to the same patient, or special instrumentation such as filoforms/followers, cystoscope or radiologic services.

Two prior U.S. patents by the present inventor; U.S. Pat. Nos. 6,599,237 and 6,994,667 are directed to some of these problems and, while they provide excellent results, they are not ideal in all applications, have some limitations in specific areas of use, and cannot therefore be considered completely versatile with respect to their application in certain surgical specialties. Another important consideration is the high cost of surgical instruments, which may be from several hundred to several thousand dollars. Some endoscopes for example may cost more than $10,000.00. Other instruments may be suited for urological use but not be suited for use in gastroenterology. Certain intubation devices such as the Councill catheter are only capable of a blind insertion and must rely on a guide wire to navigate to the bladder. Consequently, if the Councill catheter encounters resistance during insertion, there is no way to know its cause. By contrast, one aspect of the present invention is the provision of a visually directed instrument to permit continuous observation of the field just ahead of the tip of the instrument during insertion so that abnormal conditions such as obstructions or other anomalies can be continuously observed and dealt with by the physician as the instrument is being inserted. Currently, in the field of gastroenterology, intubation by means of a nasogastric tube is commonly carried out blindly or by means of a wire guide for placement into the stomach. Any obstructions, anomalous conditions, or anatomical idiosyncrasies can interfere with successful insertion of the tube. Heretofore irrigation has required an endoscope with a passage for irrigation. Moreover, no provision is made for sensing conditions at or near the distal tip of the intubation instrument with traditional analog sensors and/or actuators or smart digital sensors or actuators.

It is therefore one object of the present invention to provide surgical instrumentation for intubation that provides a sensor or multiple sensors including chemical, ultrasound, pressure, temperature sensors, or a visual sensor such as a highly versatile visually directing sensor to facilitate insertion of a catheter or other tube into the body of an animal or human patient.

Another object of the invention is the provision of a surgical instrument for visually directed intubation that is suited for use in the field of urology as well as in gastroenterology and other surgical specialties.

Yet another object is to provide a surgical intubation instrument for providing visually directed placement into the body of the patient that makes possible a dramatic reduction in the cost of the instrument.

Another object is to provide a way of permitting a medical procedure to be conducted through a catheter to protect the patient from injuries while observing a selected part of the body of the patient.

A more specific object of the invention is the provision of an improved surgical intubation instrument that allows a catheter to be routinely passed even in a difficult situation, includes a provision for enabling the patient to tolerate the catheter more readily by reducing pain and the risk of injury or infection, the elimination of many steps and procedures currently used to pass a common Foley style catheter, as well as the need for a guide wire or a filoform/follower procedure or the need for cystoscopy to pass a guide wire that is thereafter used for directing the movement of a catheter so as to reduce the frequency of complications during the insertion of a catheter.

A further object is to provide the forgoing characteristics and advantages while permitting the insertion of surgical instruments into the body without the need to remove a previously inserted catheter as well as to permit the passage of relatively large surgical instruments that cannot be inserted through an ordinary catheter.

These and other more detailed and specific objects of the invention will be better understood by reference to the following Figures and detailed description which illustrate by way of example of but a few of the various forms of the invention within the scope of the appended claims. All references listed herein are incorporated by reference to the same degree as if reproduced in their entirety herein.

### SUMMARY OF THE INVENTION

The present invention provides a method and apparatus for facilitating medical intubation procedures. In accordance with one aspect of the invention, there is provided a flexible direct vision viewing instrument or viewer that includes a catheter or sheath formed from a highly flexible biocompatible polymer such as natural or synthetic rubber or plastic having a longitudinal working channel extending the length of the catheter with an outlet port that is positioned in alignment with the channel at the distal end of the catheter. The catheter has a second longitudinal channel or lumen that contains a flexible sensor cable such as viewing cable for optical sensing. In place of or in conjunction with an optical sensor, there can be provided any of various kinds of sensors such as a chemical sensor, a pH sensor, a temperature sensor, in vivo infection, or the like. In the case of a visual sensor, one of the channels contains an optical cable providing illumination in the proximity of the distal end of the catheter for enabling the body of the patient to be viewed during placement of the instrument through a body opening or percutaneously through a surgical opening. An objective optical sensor or other sensor at the distal end of the cable provides information, e.g. continuous viewing the body of the patient just ahead of the tip of the instrument during insertion of the instrument as well as after placement of the instrument within the body. The invention is adapted to be produced in either a disposable version or a reusable version that can be sterilized after use.

The invention also provides a catheter that is able to serve as a working sheath which can be thought of as a temporary and removable artificial tract or liner that is placed through an opening in the body of the patient at the beginning of a surgical procedure to facilitate endoscopic evaluation and treatment of the digestive tract, urinary tract, or other body cavity while minimizing trauma and patient pain. During use, it allows multiple insertions and removals, i.e., the interchange of endoscopic instruments, catheters, sensors, drains, etc. The viewing cable can act as a stiffener during insertion into the patient to provide a greater degree of firmness, especially when the sheath or catheter is relatively thin or tends to fold back upon itself during insertion. Once in place, the viewing cable can be removed and replaced by other sensors such as a temperature sensor, a pH sensor, or an infection sensor, or by other medical devices. At its proximal, i.e. exterior end, the lumen of the sheath has an entry port for instruments with a removable cap that provides a nipple seal to preclude backflow of fluid from the body after the visual element or other sensor has been removed. The instrument can be placed into the stomach or other part of the digestive tract or the urethra under direct vision, i.e., with a flexible condition sensor extending through the sheath to act as a temperature, pH, or visual sensor. The sensor can include a sensor/actuator cable that provides an interoperable medium for transmitting optical or electrical signals, e.g. a fiber-optic bundle for illuminating and viewing a body cavity through the sheath, both during the insertion of the sheath and thereafter.

### THE FIGURES

FIG. 1 is a side elevational view of one form of the invention showing a viewing device at its proximal end;

FIG. 2 is a longitudinal vertical sectional view of the instrument of FIG. 1 on a larger scale;

FIG. 3 is a vertical transverse sectional view taken on line 3-3 of FIG. 2;

FIG. 4 is a vertical transverse sectional view taken on line 4-4 of FIG. 2;

FIG. 5 is an end view of the distal end of the instrument taken on line 5-5 of FIG. 2;

FIG. 5A is a partial enlarged vertical sectional view of the distal end of the instrument shown in FIG. 2 on a larger scale;

FIG. 5B is a partial enlarged vertical sectional view of the distal end of the instrument showing an objective lens built into the end of the catheter;

FIG. 6 is a side elevational view partly in vertical section showing the instrument of the invention in place within the male urethra;

FIG. 7 is a partial front elevational view of a patient showing a medical intubation appliance of the present invention connected to the patient for gastric feeding;

FIG. 8 is a side elevational view partly in vertical section to show the invention in use as a nasogastric tube;

FIG. 9 is a vertical longitudinal sectional view of the tube of FIG. 8 on a larger scale;

FIG. 10 is a transverse vertical sectional view taken on line 10-10 of FIG. 9;

FIG. 11 is an end elevational view taken on line 11-11 of FIG. 9;

FIG. 12 is a transverse sectional view showing an optional expansion feature in accordance with the invention as it appears prior to use;

FIG. 13 is a transverse vertical sectional view of FIG. 12 as it appears after being dilated by the insertion of an oversized surgical device through its central lumen; and

FIG. 14 is a schematic diagram of the viewing instrument and camera assembly.

### DETAILED DESCRIPTION OF THE PREFERRED EMBODIMENTS

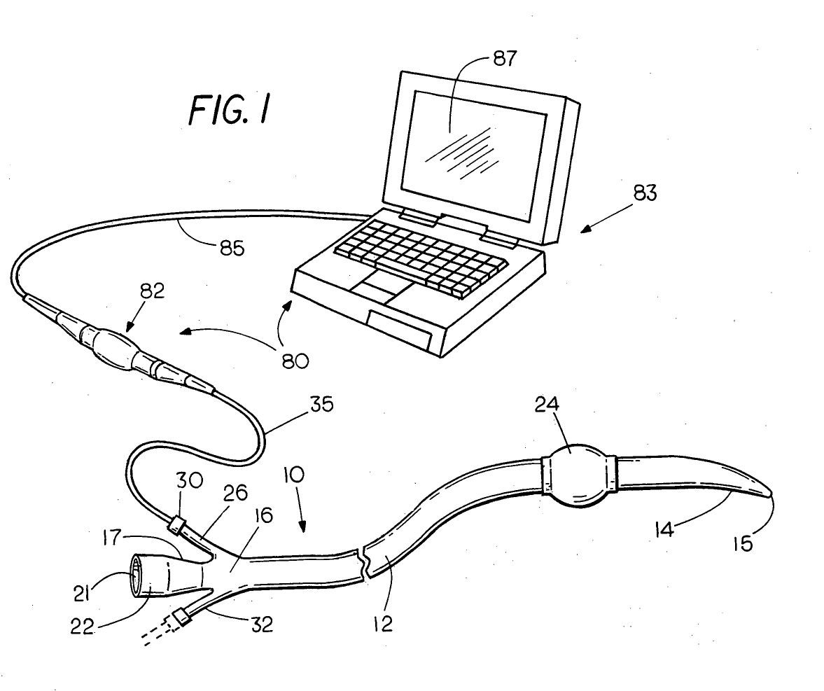

Refer now to the Figures wherein the same numerals refer to corresponding parts in the several views. The invention will be described by way of example with reference to FIGS. 1-7 and 14 which illustrate a visually directed intubation instrument in accordance with the invention that can be placed into the body of the patient under direct and continuous visual control in any of a variety of different surgical specialties. The invention is especially versatile and can be dimensioned and configured for use in urology, in gastroenterology, and in other surgical fields. The embodiment of FIGS. 1-7 and 14, illustrate the versatility of the invention since it can be employed as a drain or for exploratory purposes as well as a working channel to be used during a surgical operation or even in the field of gastroenterology as a feeding tube.

The instrument 10 comprises a flexible catheter 12 formed from natural or synthetic rubber or from a flexible biocompatible polymer of any suitable known composition such as synthetic rubber, latex rubber, polytetrafluoroethylene (PTFE), polyethylene (PE), perfluoroalkoxy (PFA), polyurethane (PU), perfluoromethylvinylether (MFA), perfluoropropylvinylether (PPVE) or other polymeric materials which would be apparent to those skilled in the art. The flexibility of the catheter 12 is apparent in FIG. 1. The catheter can also be thought of as a sheath since it is able to function in some instances as a protective sleeve for accommodating other surgical instruments that are passed through it as will be described more fully below. The catheter 12 has a proximal end 16 and a distal end 14 terminating at a tip 15. Inside the catheter 12 is a lumen that serves as working channel 18 which extends the entire length of the catheter 12 and is provided with a distal opening 18a at one end and a proximal opening 22 at the opposite end. It will be noted that the distal end 14 portion of the catheter 12 adjacent the opening 18a is tapered so that its outer diameter is progressively reduced proceeding toward the opening 18a at its tip 15. The catheter 12 can vary in length to suit the application to which it is applied, but is typically from about 30 cm to 50 cm in length and is preferably about 40 cm in length when it is to be used in gynecological procedures. It can be longer, say, 50 cm in length, when used in the male, for example, in a transurethral resection of a bladder tumor. For transurethral use, the outside diameter is typically about 9 mm (27 French) and the inside diameter about 5 mm (15 French). It should be understood that the dimensions presented herein are merely typical and can be varied to suit the circumstances in which the instrument is used. When used as a nasogastric or jejunostomy tube it can be at least 100 cm or more in length.

At the distal end 14 of the catheter 12 is provided an inflatable circumferentially extending annular balloon 24 formed from a ring of resilient elastomeric biocompatible material that extends around the catheter 12 adjacent the distal opening 18a. Inflation air or liquid is supplied to the balloon 24 when required through a tubular extension 32 at the proximal end 16 of the catheter 12 which communicates through inflation duct 33 through channel 28 with the balloon 24. If the catheter 12 is formed from an elastomer such as rubber, the balloon 24 can be integral with the sheath. However, if the catheter 12 is formed from a firm plastic material such as polypropylene, the balloon 24 is formed from rubber that is bonded to the outside surface of the catheter 12 by means of a suitable adhesive. The free end of the tubular extension 32 is provided with an inflation port through which inflation fluid (gas or liquid) can be introduced and retained until a valve, e.g. Luer lock 31 is opened.

It will be noted that the catheter 12 is provided with three channels or lumens including a lateral channel 34 that serves to accommodate the visual element, in this case a flexible fiber-optic bundle 35 for illumination and viewing, a channel 18 that can be used for drainage or as a working channel to accommodate rigid or flexible instruments that are passed through it in succession during a surgical operation, and the inflation channel 28 already described for inflating the balloon 24. It will be noted that the proximal end of the working channel 18 has an enlarged partially tapered entry port 19 with an enlarged circular open mouth 21 to give the distal end of the working channel 18 a funnel-like entry passage to accommodate the insertion of instruments during the course of a surgical operation. If desired, as shown in FIG. 3, the catheter 12 can be provided with an additional optional longitudinally extending duct 25 having an inlet port at the proximal end of the catheter and an outlet port 27 positioned at the proximal end of the balloon 24 as described in my prior patents U.S. Pat. Nos. 6,599,237 and 6,994,667 for the purpose of introducing topical anesthetics and other medicament into the passage through which the catheter has been inserted where it will be trapped between the catheter and the surrounding body tissue.

For most purposes, the fiber-optic bundle 35 is embedded within the lumen 34 of the catheter 12 so as to be fixed in place and thus not removable during the course of its useful life. However, the fiber-optic bundle can, if desired, be made removable in certain applications, for example, when the lumen 34 is used for a lavage and the central lumen 18 used for drainage. An embedded optic bundle provides a very effective yet inexpensive flexible visual catheter that can be sterilized and used repeatedly or can even be produced in a disposable form because of its low cost. This is an important feature since sterilization is expensive and sometimes may not be completely effective.

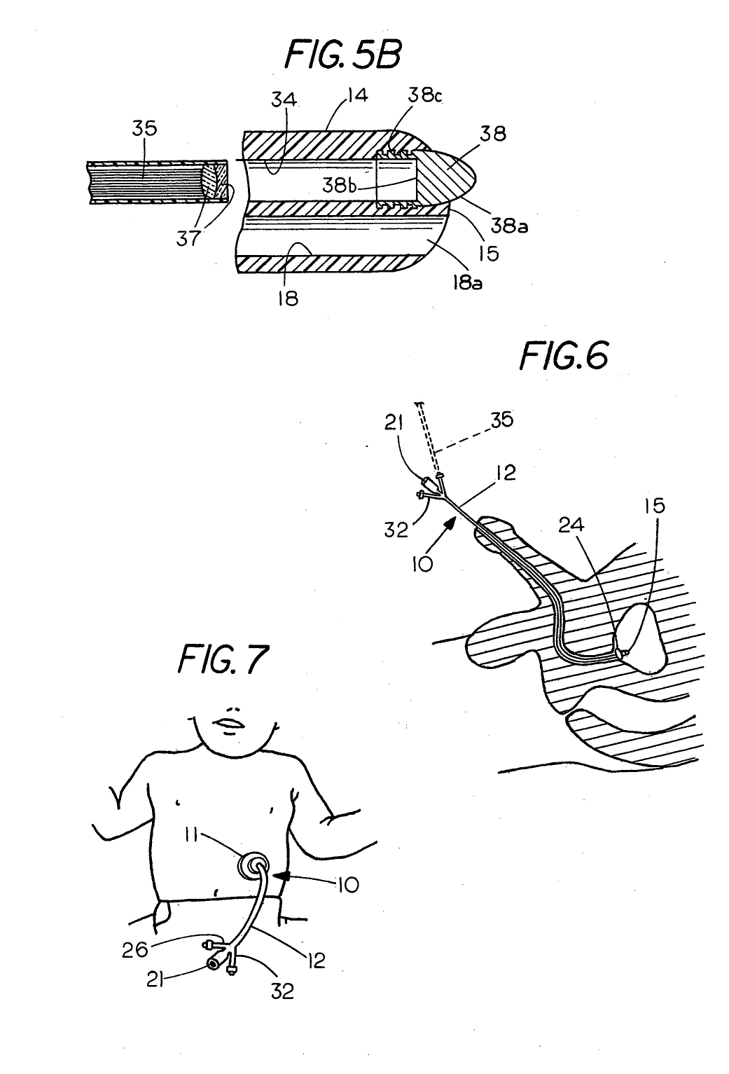

As best seen in FIG. 5A, the fiber-optic bundle 35 is provided with a viewer comprising an objective viewing element, e.g. a lens 37 that is adjacent to the opening 18a of the working channel 18. It will thus be seen that the both the objective viewing element 37 of the fiber-optic bundle 35 and the outlet port 18a of the working channel 18 face forwardly along laterally spaced apart parallel axes 39 and 41 (FIG. 5A) of which axis 39 is the optic axis of lens 37. The objective viewing element comprising the lens 37 in the embodiment illustrated projects in this case slightly beyond the free end or tip 15 of the catheter which makes wide angle viewing possible. However, if desired, for certain applications, the lens 37 can be recessed slightly within the lumen 34 so that it does not extend beyond the tip 15 of the catheter 12, but in that event wide angle viewing will be severely limited or impossible. The location of the port 18a on the end of the catheter rather than on its side allows channel 18 to be used for irrigation and other applications without the need of an endoscope for that purpose. Thus, the invention enables expensive endoscopes to be dispensed within many instances.

Refer now to FIG. 5B which shows a modified form of catheter 12 in which the lumen 34 at the tip 15 of the catheter 12 is provided with a built in, i.e. permanently attached, objective lens 38. In the example illustrated, lens 38 has a convex outer surface 38a to assure smooth passage through the urethra or other body opening and a planar inner surface 38b. The lens surfaces can, however, have any desired configuration to provide the desired optical qualities as will be apparent to those skilled in the art. To provide a secure connection, the lens 38 can be provided with an externally ribbed tubular bonding sleeve 38a adhered to the inner wall of the lumen 34 to act as a non-removable connection. One or more lenses 37 at the distal end of the optic cable 35 is selected to complement lens 38 so as to reduce or eliminate chromatic, spherical, or fisheye aberration or other possible aberration to thereby provide an integrated lens combination when the cable 35 is inserted to bring the lens 37 at its end into contact with the surface 38b of lens 38. However, if lens 38 alone provides a good image, the lenses 37 can be eliminated and the ends of the optic fibers themselves brought into contact with the lens 38 when the optic cable 35 is inserted. The lens 38 can thus provide a smoothly contoured external surface outside of and ahead of the tip 15 for achieving excellent wide angle viewing while at the same time being shaped to assure easy movement through restrictions or around obstructions. In addition, lens 38 is permanently positioned in the optimum location at the end of the tip 15 while sealing the lumen 34 to prevent the entry of fluid or other foreign material.

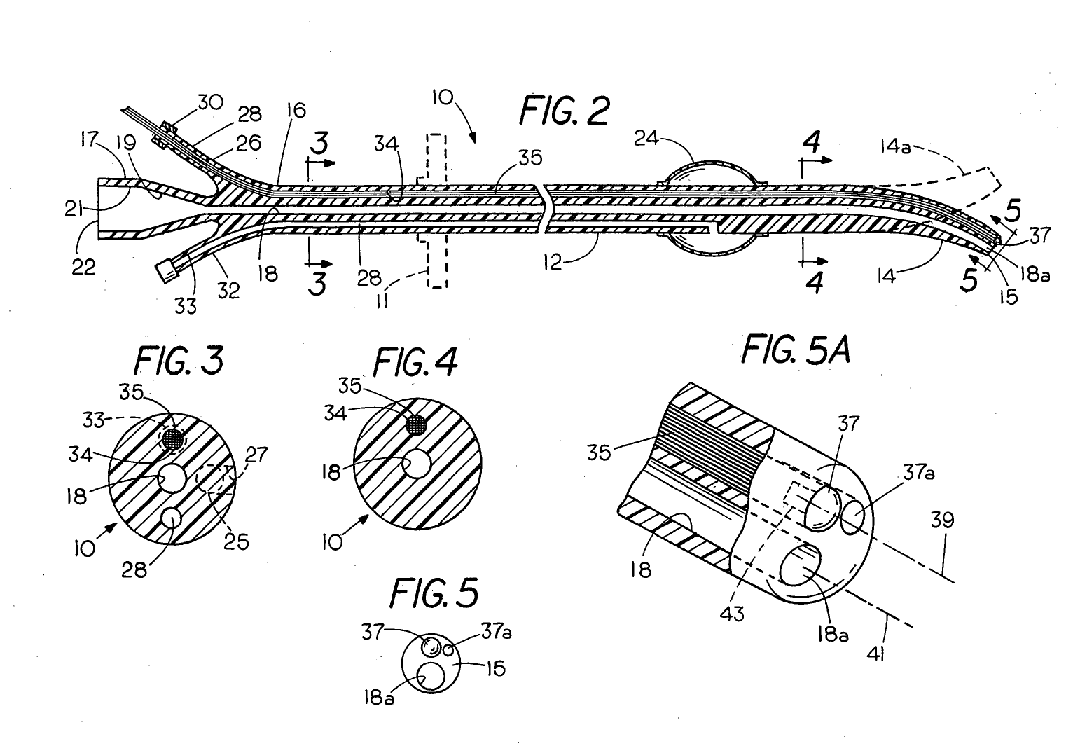

Upon encountering an obstruction during insertion, the curve shown in the tip 14 can be redirected by the operator for steering the catheter to facilitate insertion, i.e. by passive steering. The flexibility of the entire catheter including the distal end 14 is shown in FIG. 1 as well as at 14a in FIG. 2 which illustrates how the curved tip 14 can be deflected in any direction. Thus, 14a represents an alternate position of the tip as it appears when deflected upwardly or in any other direction, a feature made possible owing to the flexibility of the composite structure composed of the catheter 12 itself and the flexible visual element or cable 35.

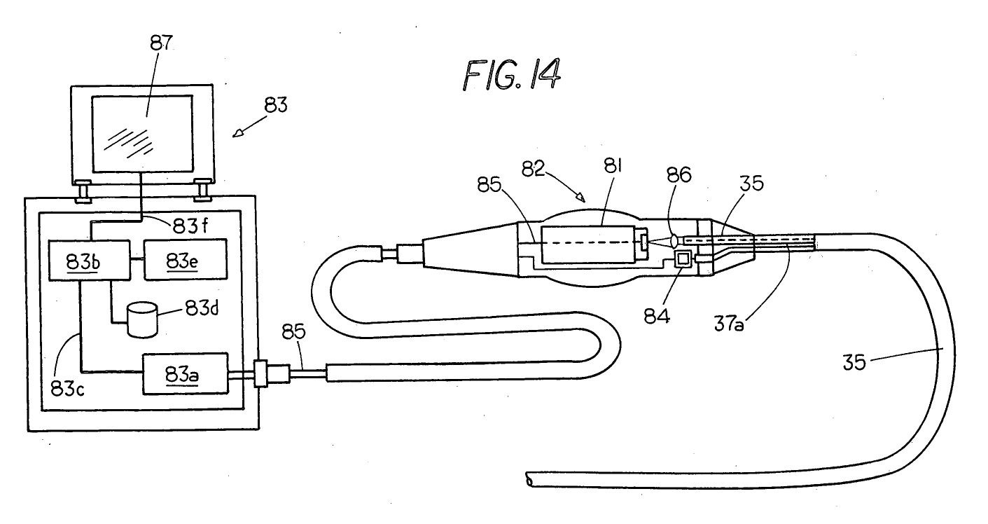

The flexible fiber-optic cable 35 which has been shown diagrammatically, can consist of crystal or glass and/or polymeric optical fibers of any suitable commercially available construction for illumination and viewing. In one preferred form, the fiber-optic bundle 35 has a fiber bundle terminating at 37a (FIG. 5A) for providing illumination from a light source 84 (FIG. 14) and a second set of fibers coupled to the lens 37 for carrying an image to a viewer or other output device 80 (FIG. 1). When cable 35 is removable, it will be seen that both the illumination fibers and imaging fibers are contained together in one removable bundle. In an alternative form, the optical fibers can be replaced by electrical conductors connected to another type of sensor such as an electronic microcamera 43 (FIG. 5A). While the medium for transmitting the optical representation of the object viewed at the tip of the catheter can be a flexible fiber optic cable made of glass or a polymeric material, when microcamera 43 is used, the medium is a flexible conductive wire consisting of copper or a pure element such as gold or silver or an alloy formulated to meet resistivity requirements, or a conductive or non-conductive liquid. For wireless transmission of video signals by radio frequency signal transmission from microcamera 43 the medium can be a pure gas or mixture of gasses or vacuum. The instrument 10 is thus provided with a sensor such as the objective viewing lens 37 focusing an image onto the microcamera 43, for example, a suitable commercially available integrated circuit having light sensitive material onto which an image is focused such as a model FSC2 camera by Schoelly GmbH of Denzlinger, Germany.

Any of several types of activators or sensors can be used for determining the state of one or more characteristics or conditions in the region ahead of or surrounding the sensor. The term “sensor” or “condition sensor” herein includes any of the following: a visual sensor, i.e. an optical viewer for producing an image, a chemical sensor including O₂, CO₂, and pH sensors, infection sensor, a pressure sensor, an audio or sonic sensor, or a temperature sensor among others. Moreover, the sensor can be a multi-sensor device which measures multiple phenomena simultaneously in real-time thus avoiding the removal of one sensor and the insertion of another sensor. Each sensor is connected to an appropriate output device 80 (FIG. 1). The output device can be a meter or oscilloscope, video display unit, or other suitable output device well known to those skilled in the art. The removal of one sensor such as an optical sensor cable following insertion, allows replacement with a different kind of sensor such as a chemical sensor or temperature sensor which is then inserted into the catheter through lumen 34 while the catheter 12 remains in place as a protective sheath within the body of the patient while sensing one or a series of different conditions or characteristics in the region ahead of or surrounding the sensor. The sensor cable can also transmit actuator signals to a proximal output instrument 83 (FIGS. 1 and 14), e.g. actuator signals for performing a predetermined function such as actuating a signal light or audible alarm (not shown) when the temperature or pH exceeds a critical level or to turn on a visual display screen, etc. The actuator can also be a valve for metering medication or anesthetic to the body tissue.

In the embodiment shown in FIGS. 1 and 14 by way of example, the fiber-optics 35 comprising glass or polymeric fibers exit the catheter at 30 to an output device 80 such as a viewing instrument which in this case is a light source and camera assembly 82 provided to receive an image from the objective lens 37 and a portable display monitor 83. The camera assembly 82 includes a miniature electronic integrated circuit camera 81 as well as a light source, e.g. the LED 84 for illuminating the area ahead of the lens 37 via fiber bundle 37a. The camera 81 is connected by electrical bus 85 to the display monitor 83 which includes a video display screen 87 for displaying the image received from the objective viewing lens 37. During operation, the image from optic cable 35 is focused by lens 86 onto electronic camera 81. While various known data display processor circuits can be employed, the display monitor 83 in this embodiment includes camera and light interface 83a feeding data to a system control 83b via bus 83c which is coupled to data storage unit 83d and to a data acquisition and processing center 83e. The system control 83b feeds data to LCD color monitor 87 via bus system 83f in video format for displaying an image of the patient's body. One suitable electronic camera and light source assembly 82 is FSC2 (FlexiScope 2). The signal from bus 85 can also be routed to digital output ports (not shown) to display the image on a local color monitor or for streaming the video over the Internet. If the signal is to be stored for future use, the video signal is processed through a computer hard drive for storage. The invention makes possible the continuous display of the image of the body passage obtained from lens 37 in real time as the catheter 12 is being inserted so that any discontinuities or obstructions can be observed and circumvented during the insertion procedure. Following insertion, an image of the urinary tract, gastrointestinal tract, or other body cavity that has been entered can be observed. If a sensor other than an optical sensor is used, the condition being sensed, e.g. the temperature, chemical composition, pH, etc. at the distal tip 15 of the instrument can be monitored on a suitable output device, e.g. meter or oscilloscope, etc. that is used in place of the display monitor 83. If desired, the microcamera 43 (FIG. 5A) can also include a radio signal transmitter for transmitting a signal depicting or representing a condition or a visual image, in which case the radio transmission sent to the output device replaces the electrical bus 85 and fiber-optics 35 which are then eliminated. The cable 35 is “resposable” after use, i.e. it can be pulled out of the catheter 12, cleaned and resterilized, inspected for functionality, then inserted into a new and sterile catheter. It is then inspected to determine that it is functioning properly and is ready for its next use. The catheter 12 is intended to be disposed of after each use. After a specified number of uses, the cable 35 is also disposed of. The cable 35 is preferably compatible with standard sterilization techniques such as EtO (ethylene oxide), glutaraldehyde, Steris, Sterrad sterilization or other industry standard sterilization techniques.

The transmission cable 35 as already mentioned can also be embedded in the catheter 12. The term “embedded” or “non-removable” herein is intended to mean that the cable 35 whether it be fiber-optics or an electrical cable is mounted securely enough so that it is not meant to be removed or easily removed in a simple manner by the user, although it is apparent, however, that it might be possible for a person to remove even an embedded cable with sufficient time and effort. The embedded cable can be held in place either mechanically. For example by means of surface irregularities which are gripped by the surrounding rubber of the catheter 12, or by being bonded in place within the passage 34, i.e. held in place by adhesion as the rubber or other flexible polymer forming the catheter 12 is cured. During manufacture, a cable 35 can be inserted into the passage 34 after the catheter has been completely formed then bonded in place or, if desired, it can be molded in situ as the catheter is being molded and before the polymer is cured or otherwise fixed within the catheter in any other manner known to those skilled in the art.

It is important to note that both the cable 35 and the catheter 12 are highly flexible so that together they form a composite structure which can flex in any direction as it is being inserted. This is especially advantageous during a difficult passage or through a curved duct such as the male urethra or when an obstruction is encountered. Flexing of the entire catheter is illustrated in FIG. 1. Flexing of the distal tip 14, e.g. to alternate position 14a is shown in FIG. 2 so as to enable the instrument to bend around corners or dodge obstructions. The cable 35 can also add a degree of stiffness to the instrument 10 so that sufficient stiffness is provided to ensure that the entire instrument consisting of the catheter 12 and cable 35 can be easily inserted even through a tight passage, e.g. through the urethra without buckling, a problem sometimes referred to as a “wet noodle” effect wherein the entire instrument buckles as an axial force is applied to the proximal end by the operator in an attempt to push the distal end around a curve, past an obstruction or under other circumstances where resistance is encountered. If desired, to provide additional stiffness, the cable 35 can be enclosed in a tubular casing 33 (FIG. 3) enabling it to serve as an obturator having a predetermined stiffness which makes the instrument 10 less subject to the possibility of buckling when axial pressure is applied.

Refer now to FIG. 6 which illustrates how the catheter 12 is inserted into the male urinary tract to allow examination of the urethra and the bladder. It will be noted that the catheter 12 is able to easily flex so as to negotiate curves in the urethra without difficulty and as the instrument is being inserted, the image just ahead of the distal end of the instrument can be continuously observed while noting pathological conditions or abnormalities in case the insertion becomes difficult or an obstruction is encountered. If the optical cable 35 is embedded, i.e. fixed in the catheter 12, it remains in place following insertion thereby making continuous observation possible. The working channel 18 which can be temporarily plugged by means of a cap or other seal (not shown) is then opened at its proximal end to allow one or several successive instruments to be introduced through the open mouth 21 as required during a surgical operation by passing them through the channel 18 into the bladder or other organ while the catheter 12 remains in place, thereby serving as a protective sheath in the manner described in my prior patents U.S. Pat. Nos. 6,599,237 and 6,994,667 to prevent injury to the patient. The present invention however, has the added benefit of permitting visual observations to be made continuously via the optic cable 35 while the working channel 18 (FIG. 2) is used contemporaneously for drainage, for the passage of instruments used in surgery, or for any other purpose.

An important feature of the invention is ability of any channel (channel 18 or 34) to be used for irrigation of the bladder or other organ, whereas heretofore an endoscope was required for this purpose. The invention, besides providing visualization, thus allows irrigation to be performed without the need for an expensive endoscope. Once the instrument 10 has been completely inserted, the balloon 24 is inflated by introducing a fluid or gas through the passage 28 to hold the catheter 12 in place.

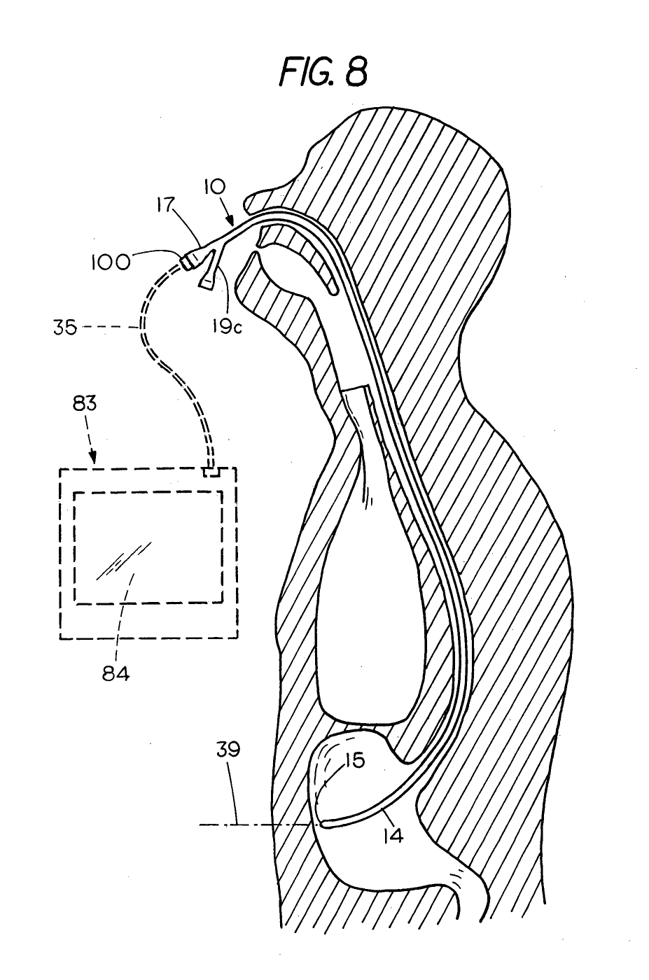

Refer now to FIG. 7 which illustrates how the invention can be used in gastroenterology, in this case as a gastronomy/gastrostomy tube that serves as a gastric feeding tube. When used as a gastric feeding tube, the instrument 10 is preferably provided with an abdominal mounting disc 11 which is bonded conventionally to the outside wall of the abdomen to hold the instrument which is inserted percutaneously in place where it enters the abdomen through the skin. The tube 10, which can be referred to as a percutaneous endoscopic gastrostomy tube, provides a convenient visually directed access route for the delivery of long-term enteral nutrition through the stomach. It is surgically placed in the abdominal wall as shown in FIG. 7 below the rib cage and slightly to the left in this case for feeding an infant. The optic cable 35 or other condition sensor permits continuous visual or non-visual monitoring both during insertion and following insertion. When used as a feeding tube as shown in FIG. 7, the catheter 12 is held in place by means of the inflated balloon 24 as well as sutures, if desired.

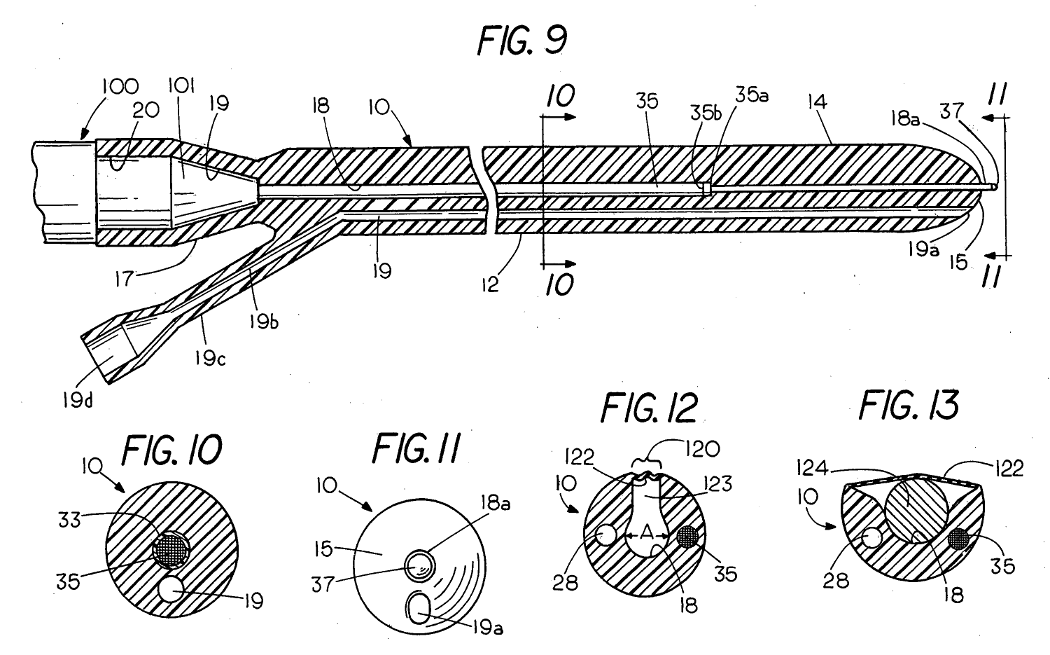

Refer now to FIGS. 8-11, which illustrate a visually directed nasogastric tube in accordance with the invention wherein the same numerals refer to corresponding parts already described. In this embodiment, the flexible optical cable 35 is connected at 100 to a viewing instrument 83. In this embodiment, the cable 35 includes a tapered barrel 101 that fits into a tapered socket 19 within the catheter 12. As described earlier, a light source is provided to which the optic cable 35 is connected. In FIG. 8, a light source and camera assembly (not shown) similar to 82 of FIGS. 1 and 14 is provided within monitor 83 for directing light into the fiber-optic bundle 35 and out through the lens 37 to illuminate the field just ahead of the tip 15 of the instrument 10. The image proximate the lens 37 is then carried back through the fiber-optic bundle 35 to the monitor 83 and viewing screen 84. Cable 35 extends from port 21 at the proximal end of the channel 18 to the distal end 14 and as shown in FIG. 9 for most purposes projects slightly beyond the tip 15 of the catheter 12. With the objective viewing lens 37 located just beyond the tip 15 of the catheter, enhanced viewing ahead and also to the side is made possible by the wide angle of view that is permitted both while the catheter 12 is being inserted as well as after it is in place within the body of the patient. In the nasogastric tube of FIGS. 8-11, the fiber-optic bundle 35 is preferably removable.

As shown in FIG. 9, the cable 35 has a distal segment of reduced diameter which can be any length, e.g. 2-3 inches long to define a shoulder 35a in the cable so as to provide a proximal portion having a relatively large diameter and a distal segment of a reduced diameter with a shoulder between them which acts as a retainer. The channel 18 in the catheter is shaped like the cable 35. Thus, when the cable is fully inserted, the shoulder 35a rests against a similarly shaped restriction in the channel 18 which serves as a retainer or stop to check the distal movement of the cable. In a preferred form, a circular washer 35b of a selected thickness and having an outside diameter the same as the larger diameter of the cable 35 is mounted on the cable at the shoulder 35a to act as a retainer for determining the position of lens 37 relative to the tip 15 of the catheter 12 during use to thereby control the extension, if any, of lens 37 beyond the tip 15.

FIG. 9 thus shows a removable transmission cable 35 slideably mounted within a channel 18 as well as a working channel 119 positioned laterally of the channel 18. Channel 119 has an outlet port 119a at the distal tip of the instrument just below the outlet port through which the cable 35 extends. The proximal end of the working channel 119 extends at 119b through a proximal extension 119c terminating at an opening 119d through which fluid can be drained from the body or surgical instruments can be passed when required through the catheter 12 into the patient. The fibers within the cable 35 can be enclosed within a tubular casing 33 (FIG. 10) to hold the fibers together. During use, as shown in FIG. 8, the visually directed instrument 10 comprising a nasogastric tube can be held in place conventionally where it enters the nose with adhesive tape (not shown) and accordingly no balloon is required for holding the tube in place or within the body. The viewing instrument 100 as shown in FIG. 8 is connected by means of a cable 35 to the visual display 83 which includes the video display screen 87 for continuously displaying in real time an image of the area just ahead of the distal tip 15 of the instrument.

Instrument 10 comprising the visually directed nasogastric tube is used for patients who are unable to ingest nutrients by mouth and is inserted through either nostril and passed down through the pharynx and esophagus into the stomach, typically for short-term feeding. Placement must be checked before each feeding. This can be done by viewing the area just ahead of the tip 15 by displaying it on the viewing screen 87. Another use for the nasogastric tube is to drain accumulated fluids from the stomach and small intestine due to a blockage of the bowel from an obstruction or bowel inactivity. The present invention is particularly advantageous in overcoming the problems that resulted previously from the conventional feeding tube curling up in the esophagus, becoming diverted into the trachea, or coming to rest in a less than optimal location in the stomach. When these problems arose prior to the present invention, the solution was to take a static x-ray (using abdominal film) or measure the presence of CO₂ to rule out placement of the tube in the trachea. These procedures were complicated and took time since it was necessary to move the patient to the radiology department or transport x-ray equipment to the patient's room for the x-rays, adjust the tube, then take additional x-rays to verify the actual location of the tube and, of course, a radiologist is required to read the x-rays.

The visually directed nasogastric tube in accordance with the invention thus has two lumens; channel 18 in which the visual element or cable 35 is preferably removably mounted and the working channel 119, which serves as the primary working channel for drainage and/or feeding. However, if the visual element 35 is removed, channel 18 can also be used as a working channel, for example, to pass an instrument or succession of instruments through the catheter 12 into the body of the patient. Consequently, the invention provides continuous visually directed insertion of the catheter while also providing, if desired, a pair of parallel laterally spaced apart working channels that can each be used as a working channel for different purposes during surgery or convalescence. For example, channel 18 can be used for drainage while at the same time the channel 119 is used for inserting and removing a variety of surgical instruments or guide wires through the catheter which then acts as a protective sheath that reduces discomfort, eliminates pain that would otherwise be experienced, and the tissue trauma that would occur if the instruments were passed directly through a body opening without the catheter 12 in place. Channel 18 which is preferably the largest in diameter is well suited for drainage and/or feeding the patient. When the visual element 35 is removable, it is preferably enclosed within the flexible protective plastic casing 33 and coated on the outside with a suitable surgical lubricant so that it can be removed when desired from the instrument 10. The visual element 35 and casing 33 also provides a degree of stiffness for the catheter 12 so that it can be reliably pushed through a tight passage and yet is able to flex freely around and through curved body openings and easily pass obstructions. In such a case, the visual element acts to assist in insertion and thus serves as an obturator for adding a degree of stiffness to the catheter.

It will be thus understood that the invention provides continuous visually directed placement as well as allowing the position of the distal end of the instrument to be confirmed by the operator at the time of placement. Consequently, it eliminates the need for x-rays and the services of a radiologist to read them as well as the need for a CO₂ determination procedure. As already described in connection with FIGS. 1-7, in place of a visual sensor, the invention can employ any other known form of sensor for evaluating one or more conditions along the length of the cable 35 or in the region just ahead of the tip 15 of the instrument, e.g. a chemical sensor, a temperature sensor, a pressure sensor, etc.

To more fully explain the invention and the results that can be achieved, an additional example will be presented to illustrate its capabilities. Once the instrument 10 comprising the nasogastric tube (FIGS. 8-11) is in place within the stomach, the visual element 35 and the light beam on axis 39 provided by the light source 84 permits the doctor to identify the exact location for retrograde placement of a percutaneous guide wire, that is to say, where a hole is to be punched with the guide wire from the outside of the patient through the skin of the abdomen into the stomach while being guided by the light within the stomach that is directed as a beam through the lens 37. The light transmitted along the optic axis 39 (FIG. 8) at the tip 15 of the instrument 10 comprising the nasogastric tube is bright enough for the doctors to see it by transillumination through the skin when observing the patient from the exterior. The beam can be positioned conventionally by guide wires (not shown) in the catheter 12 as described in patent U.S. Pat. No. 6,994,667. The doctor can then choose to insert the guide wire from the inside out (antegrade) through the lateral working channel 119 while the light is on, or from the outside in retrograde, whichever is preferred. If the retrograde procedure is used, the guide wire is inserted from the exterior of the body through the skin into the stomach at the exact location of the light transmitted from the tip 15 of the instrument along the axis 39 (FIG. 8). Thus, the visual element of 35 of the instrument 10 comprising the nasogastric tube allows the doctor to place the guide wire precisely. The instrument 10 comprising the nasogastric tube is then used as a working channel device to pull the guide wire and/or feeding tube of FIG. 7 into the stomach via the working channel 119. On the other hand, in the antegrade procedure, the doctor is assisted by the light from the visual element to correctly pass the guide wire from the stomach out through the skin of the abdomen.

Refer now to FIGS. 12 and 13 which illustrate a modified form of the invention in which the catheter 12 is provided with a longitudinally extending area designated 120 running throughout the length of the catheter that has a reduced wall thickness which is bridged across by a stretchy elastic sheet or band 122. The reduced wall thickness can be seen in FIG. 12 as a gap 123 adjacent band 122. During use, when the catheter 12 is in a relaxed resting state as shown in FIG. 12, the lumen 18 has a predetermined diameter A capable of accommodating surgical instruments of a certain size that are to be passed through it. However, as shown in FIG. 13, when a surgical instrument 124 of a much larger size is passed through the lumen 18, the elastic band 122 that covers the area of reduced wall thickness, the band 122 becomes stretched as the wall of the catheter 12 is extended by the instrument 124, thus allowing surgical instruments 124 of a much larger size than the initial diameter of lumen 18 to be passed through the catheter 12 and into the body of the patient for carrying out various surgical procedures, e.g. cauterization, tumor removal, or for other purposes. The invention thus provides an expansion zone 120 extending the length of the catheter 12 that is bridged by the relatively thin elastic expansion band 122 so as to allow enlargement of the lumen 18 along the entire length of the catheter 12 for introducing or removing instruments 124 that are larger than the lumen 18.

The band 122 over the thin wall area at 120 thus provides a catheter having a greatly expandable lumen 18 yet which maintains its integrity, i.e. lumen 18 does not open out into the body passage or communicate with any other part of the body except through the opening at the distal tip 15. The catheter is therefore able to expand substantially to enable oversize instruments such as that shown at 124 to be passed into the body, yet the wall of the body opening is protected at all times by the catheter and the elastic band 122 so as to avoid injury that might otherwise be induced by the instrument 124 as it is being inserted or retracted.

Many variations of the present invention within the scope of the appended claims will be apparent to those skilled in the art once the principles described herein are understood.

What is claimed is:

1. A medical intubation instrument for placement into an animal or human patient comprising, a catheter having a proximal and a distal end and an elongated body formed from flexible biocompatible polymeric material including a longitudinal working channel extending the length thereof with an outlet port that is positioned in alignment with said working channel so as to be located on the distal end of the catheter, said catheter having a second longitudinally extending channel containing a flexible sensor cable with a sensor connected thereto for sensing at least one characteristic or condition in the region ahead of or surrounding the sensor, such that the composite structure comprising the cable and catheter together is able to flex freely so as to enable the instrument to bend for negotiating a curved entry passage or to circumvent an obstruction while the sensor is in operation for determining one or more of said characteristics or conditions within the body of the patient.

2. The intubation instrument of claim 1 wherein the sensor cable is embedded within the catheter so as to be non-removable therefrom.

3. The instrument of claim 1 wherein the sensor cable is slideably mounted within the second channel extending through the catheter for enabling the sensor cable to be removed therefrom.

4. The instrument of claim 1 wherein the sensor cable comprises a visual sensor and illumination cable and the sensor comprises an objective viewing element to provide an image of the body of the patient adjacent the location of the sensor on the instrument.

5. The instrument of claim 1 including an inflatable balloon proximate the distal end of the catheter for retaining the instrument within the body of the patient.

6. The instrument of claim 1 wherein the catheter has a longitudinally extending area of reduced thickness in the wall thereof comprising an expansion zone that is bridged by an elastic expansion band to allow enlargement of the working channel along the length of the catheter to accommodate the insertion therethrough of one or a succession of surgical instruments that are larger than the working channel through which they are passed.

7. A flexible visually directed composite medical intubation instrument for placement into an animal or human patient comprising, a catheter body having a proximal and distal end and being formed from a flexible biocompatible polymeric material that has a longitudinal working channel extending the length thereof with an outlet port that is in alignment with the working channel so as to be located at the distal end of the catheter, said catheter having a second longitudinally extending channel containing a flexible optical cable for providing illumination proximate the distal end of the catheter to enable the body of the patient to be continuously viewed as a composite instrument that comprises the combined cable and catheter is able to flex freely during placement of the instrument through an opening in the body or percutaneously through a surgical opening and an objective optical element coupled at the distal end of the cable for continuous viewing of the body of the patient during insertion of the instrument and after placement of the instrument has been completed.

8. The instrument of claim 7 wherein the optical cable is slideably mounted for removal in place of or in conjunction with a lumen of the instrument for replacement by a sensor comprising a chemical sensor, a pH sensor, a pressure sensor, an audio sensor or a temperature sensor or an infection sensor.

9. The intubation instrument of claim 7 wherein the cable is embedded in the body of the catheter so as to be fixed in place therein.

10. The instrument of claim 7 wherein the optical cable is slideably mounted within the catheter to allow replacement thereof by the insertion of one or more sensors for sensing different conditions within the body of the patient, such sensors being adapted to be introduced and removed in succession through said channel to thereby sense a plurality of different characteristics or conditions within the body of the patient while the catheter remains in place as a protective liner within the body of the patient.

11. The instrument of claim 7 wherein both the objective viewing element and the outlet port of the working channel are located at the distal end of the catheter.

12. The instrument of claim 11 wherein both the objective viewing element and the outlet port of the working channel face forwardly along laterally spaced apart axes that extend distally from the distal tip of the catheter and the flexibility of a composite structure comprising the flexible cable and catheter enables the catheter including a distal end portion thereof to flex in any direction for negotiating curves and circumventing obstructions as the optic axes of the viewer and the adjacent axis of the outlet port of said working channel are deflected simultaneously in the same direction.

13. The instrument of claim 7 including a passage having an outlet port proximate the distal end of the catheter for introducing medicament into the body of the patient.

14. The instrument of claim 7 wherein the sensor cable comprises a fiber-optic cable and a sensor comprises an objective viewing lens affixed at the distal end of the cable for wide angle viewing to provide a wide angle image of the body of the patient ahead of the distal end of the instrument.

15. The instrument of claim 7 including an inflatable balloon proximate the distal end of the catheter for retaining the instrument within the body of the patient.

16. The instrument of claim 7 wherein the catheter has a longitudinally extending area of reduced thickness in the wall thereof comprising an expansion zone that is bridged by an elastic expansion band to allow enlargement of the working channel along the length of the catheter for accommodating the insertion therethrough of one or successive surgical instruments that are larger than the working channel through which they are passed.

17. A flexible visually directed gastric tube for placement into the stomach or other part of the digestive tract of an animal or human patient comprising, a catheter body having a proximal and distal end and being formed from a flexible biocompatible polymeric material that has a longitudinal working channel extending the length thereof with an outlet port that is in alignment with the working channel so as to be located at the distal end of the catheter, said catheter having a second longitudinally extending viewing channel containing a flexible optical cable to provide illumination proximate the distal end of the catheter that enables the body of the patient to be continuously illuminated during insertion of the instrument through an opening in the body or percutaneously through a surgical opening and an objective optical viewer at the distal end of the cable for viewing a portion of the stomach or other part of the digestive tract of the patient during insertion of the instrument and after placement of the instrument has been completed.

18. The gastric tube of claim 17 wherein the optical sensor comprises an objective viewer and a cable that includes a fiber-optic bundle for illuminating and for viewing the image of the patient through said objective optical viewer.

19. The apparatus of claim 18 wherein objective viewer comprises a microcamera to transmit a signal to a viewing instrument for displaying an image of a portion of the body of the patient proximate the microcamera.

20. The apparatus of claim 19 wherein the microcamera is connected to the viewing instrument by an electrical conductor extending through said cable.

21. The apparatus of claim 19 wherein the microcamera includes a radio signal transmitter for transmitting an image of a portion of the body of the patient to the viewing instrument for being displayed thereby.

22. The apparatus of claim 17 wherein the instrument comprises a visually directed flexible percutaneous endoscopic gastrostomy tube that provides access adapted for the delivery of short or long-term enteral nutrition to the patient through the wall of the abdomen.

23. A method for facilitating the sensing of a characteristic or condition within the body of an animal or human patient comprising the steps of, providing a flexible catheter having a proximal and distal end, said catheter comprising an elongated body formed from flexible biocompatible polymeric material, providing a longitudinal working channel that extends the length of the catheter and has an outlet port positioned in alignment with the working channel so as to be located on the distal end of the catheter, providing said catheter with a second longitudinal channel with an outlet port that is adjacent the port of the working channel, inserting at least one flexible sensor cable having a sensor thereon by sliding the cable into the second channel to an operating position, sensing a characteristic or condition proximate the sensor while the working channel remains available for draining fluid, for irrigation, for feeding the patient or for passing one or a succession of surgical instruments therethrough into the body of the patient and the flexibility of the composite structure comprising the cable and catheter enables the instrument to negotiate curves and circumvent obstructions during said insertion thereof into the body of the patient.

24. The method of claim 23 including the step of providing as said flexible sensor cable at least one member selected from the group consisting of a visual sensor for optical viewing, a chemical sensor, a pH sensor, an infection sensor, a pressure sensor, an audio sensor, and a temperature sensor.

25. The method of claim 23 including the steps of removing said sensor from the catheter and thereafter inserting at least one additional sensor cable through said channel for sensing a different condition within the body of the patient.

26. The method of claim 23 wherein the catheter is a nasogastric tube and including the steps of, passing the tube into the stomach of the patient, providing a light beam at the distal end of the catheter for visibly illuminating a portion of the skin of the abdomen of the patient from inside the stomach, moving the instrument to change the illuminated portion of the patient's skin to a selected position and placing a guide wire for retrograde or antegrade passage through the abdomen in the position determined by the selected position of the light beam.

27. The method of claim 23 including the step of passing a catheter over a guide wire into the stomach of the patient.

28. The method of claim 23 including the step of introducing medicament through the catheter into the body of the patient at a location positioned between the catheter and surrounding body tissue of the patient.

29. The instrument of claim 1 wherein the sensor cable is connected to an output device and for transmitting an actuator signal to the output device to actuate the device for performing a predetermined function.

30. The instrument of claim 3 wherein the sensor cable has a portion with a relatively large diameter and a distal segment of a reduced diameter with a should therebetween that fits a similarly shaped portion of the second channel to act as a retainer so as to check the distal movement of the cable for locating the distal end thereof in a predetermined position.

31. The instrument of claim 30 wherein the shoulder of the cable positions the distal end of the cable beyond the distal tip of the catheter.

32. The combination of a medical catheter with a built-in optical viewing device for placement into an animal or human patient comprising, a catheter having an elongated body formed from flexible biocompatible polymeric material including a longitudinal working channel extending substantially from a proximal end to a distal end thereof, said working channel having an outlet port that is positioned at the distal end of the catheter for communicating with the interior of the patient's body proximate the distal end of the catheter, said catheter containing a flexible optic cable that is non-removably embedded therein and extends substantially the length therethrough alongside the working channel, said cable having an objective viewing element to provide an image of the region ahead of or surrounding the viewing element, such that the composite structure that comprises the cable and catheter together is able to flex freely so as to enable the composite structure to bend while negotiating a curved entry passage or circumventing an obstruction with the viewing element in operation for providing an image within the body of the patient as the catheter is being inserted therein, such that the working channel is available for drainage; for irrigation or for the passage of medical instruments therethrough while the patient is viewed through the optical cable.

33. The instrument of claim 1 including a camera assembly operatively connected thereto, said camera assembly comprising a light source for illuminating an area proximate the distal end of the catheter, a camera positioned in optical alignment to receive an image from the sensor cable such that an image carried by the sensor cable is directed onto the camera and an electrical conductor is connected between the camera and a display device for displaying an image received from the camera.

34. The instrument of claim 7 including a camera assembly operatively connected thereto, said camera assembly comprising an electronic camera positioned in optical alignment to receive an image from the sensor cable, a light source for illuminating a selected area proximate the catheter and an electrical conductor connected between the electronic camera and a display device for displaying an image of the area illuminated by the light source.

35. The instrument of claim 32 including a camera assembly operatively connected thereto, said camera assembly comprising an electronic camera positioned in optical alignment with a proximal end of the sensor cable to receive an image from the digital end of the sensor cable, a light source adjacent the proximal end of the sensor cable for illuminating a selected area proximate the distal end of the catheter and an electrical conductor connected between the electronic camera and a display device for displaying an image of the selected area illuminated by the light source.

36. The apparatus of claim 1 wherein an objective lens is bonded to the catheter at a distal end of the second channel and the second channel is adapted to contain an optical cable positioned in optical alignment with the objective lens to receive an image of the patent therefrom.

37. A method of using a surgical instrument for facilitating examination or for performing a surgical operation on the body of a patient comprising,
providing a flexible working sheath for insertion into a body cavity at the beginning of a surgical procedure,
providing said sheath with an elongated tubular body having a first longitudinal lumen of sufficient size to accommodate elongated sensing devices or other medical devices that are inserted through the lumen of the sheath into the body of a patient, said first lumen having an opening at a proximal and distal end thereof,
providing a second lumen in the sheath that is open at both ends thereof for irrigation or to act as a drain,
placing a removable viewing cable in the first lumen of the sheath to provide stiffness for the instrument so as to facilitate the insertion of the working sheath into the body of the patient,
placing said instrument during use percutaneously or through a body passage into a body cavity within the body of the patient and allowing the instrument to remain substantially stationary until the examination or procedure is complete, such that while remaining in place the viewing cable is removed from the working sheath and one or more of the said sensing devices or other medical devices is passed through the first lumen into the body through the sheath,
said sheath thereby serving as a single insertion instrument which provides an artificial protective lining in a body opening through which it is passed to minimize tissue trauma and discomfort or pain for the patient during the passage or said devices therethrough while the second lumen enables fluid to be transferred to or from the patient.
